(19) United States
(12) Patent Application Publication
(10) Pub. No.: US 2015/0344934 A1
(43) Pub. Date: Dec. 3, 2015

(54) Novel High Throughput Assay for finding new Jak3 Interacting Compounds, Biomolecules, and Inhibitors.

(71) Applicants: Narendra Kumar, Kingsville, TX (US); Jayshree Mishra, Kingsville, TX (US)

(72) Inventors: Narendra Kumar, Kingsville, TX (US); Jayshree Mishra, Kingsville, TX (US)

(21) Appl. No.: 14/483,622

(22) Filed: Sep. 11, 2014

Related U.S. Application Data
(60) Provisional application No. 61/960,652, filed on Sep. 23, 2013.

(51) Int. Cl. C12Q 1/48

(52) U.S. Cl. CPC C12Q 1/485; G01N 2500/04

(57) ABSTRACT

Janus Kinase 3 is a non-receptor tyrosine kinase that mediates signals initiated by cytokines through interactions with the receptors of cytokines. Abnormal activation of Jak3 was associated with human hematologic and epithelial malignancies. Inhibitors of Jak3 have shown utility in many different disease such as autoimmune disorders, allograft rejection during transplantation, acute lymphoblastic leukemia, Type 1 diabetes, rheumatoid arthritis and allergy and asthma. Since these inhibitors make their way into clinical trials with profound effects, it is essential to develop a sensitive, precise, and rugged screening tool to screen synthetic compounds or other biomolecules that has the potential to modulate Jak3 functions and hence treat a wide variety of diseases. Present invention relates to novel high throughput system for finding previously unknown Jak3 interacting compounds, human biomolecule (e.g. proteins or others), and those compounds that can inhibit Jak3 activations. All these identified compounds and molecules can be used for the treatment of a wide variety of diseases (listed in table 1) where Jak3 activations or its interactions with other biomolecules are essential for disease sustenance or propagation in human body.

### BACKGROUND

Janus Kinases (Jaks) are a family of non-receptor tyrosine kinase with four members; Jak1, Jak2, Jak3, and Tyk2. Like other members, Jak3 mediates signals initiated by cytokine through interactions with receptors for IL-2, IL-5, IL-7, IL-9, and IL-15 via the common γ (gamma) chain of these receptors (1). Different studies showed that Jak3 is widely expressed in different organs of both humans and mice (1-3). Abnormal activation of Jak3 was associated with human hematologic and epithelial malignancies (4,5). Inhibitors of Jak3 have shown utility in many different autoimmune disorders, including allograft rejection during transplantation, acute lymphoblastic leukemia, Type 1 diabetes, rheumatoid arthritis and allergic and asthmatic diseases. These inhibitors are making their way into clinical trials with profound effects, thus it is of significant importance to develop a sensitive, precise, and rugged screening tool to screen synthetic compounds or other biomolecules that has the potential to modulate Jak3 functions and hence treat a wide variety of diseases.

Currently available screening tools use only the kinase domain of Jak3 and use among others mainly two formats viz. (a) Caliper format and (b) Perkin Elmer format. Calipers's mobility shift assay (Caliper Life Science, Hopkinton, Mass.) uses a nanofluidisc-based technology that involves electrophoretic separation of fluorescently labeled substrate (phosphorylated and non-phosphorylated) on a microchip and fluorescence intensity of each is measured. Additionally the reactions are also run on a chip or in a microplate wells with microchip being used solely as separation device between the substrate and the product. The main limitation of this technology is the use of external peptide substrate and the need to screen an efficient substrate that can fit into this technology. Screening of these substrates gives another layer of complexity and cost-intensiveness. Another well know limitation of fluorescence based assays are their sensitivity towards both quenching and fluorogenic compounds. Though mobility-shift technology mitigates this problem to some extent, however this adds substantially to the cost of screening vast array of compounds including time-delay, instrument-, and manpower-cost. Another technology is Perkin Elmer (Waltham, Mass.) Streptavidin coated Flashplate radiometric assay, which uses radio-labeled external substrate and cost-intensive instrument. This method uses one of the three ways to determine phosphorylation: measuring ATP depletion (easylite-Kinase™), direct measurement of phosphate incorporation by the substrate using 33P-labeled ATP (FlashPlate®, filter-binding), or capture and measurement of phosphorylated substrate (AlphaScreen®, LANCE™, DELFIA®). Both these assay uses only kinase domain of Jak3. This necessitates the use of external substrate with costly and less user-friendly methods of either detection or separation. Another technology by Dynamii Pharmaceticals, DynamixFit™ also uses the kinase domain but utilizes an “induced fit” concept different from the classical ‘lock and key model’ of enzyme activity. Since enzymes are flexible proteins, the 3D structures of their active sites are continuously reshaped by interactions with their substrates (or inhibitors), a process called ‘induced fit’. Overall, all currently available screening tools have several common deficiencies including low sensitivity, requiring an external substrate, and requiring a radioactive detection.

### SIGNIFICANCE OF THE PRESENT INVENTION

Overall either deficiency or over-activations of Jak3 both leads to different immunological disorders such as inflammatory autoimmune diseases, rheumatoid arthritis (RA), psoriasis, transplant rejection and other allergic responses (6-8), and different cancers (9-11). So far as cancer alone is concerned, there were an estimated 14.1 million cancer cases around the world in 2012, of these 7.4 million cases were in men and 6.7 million in women. This number is expected to increase to 24 million by 2035 (www.wcrf.org). Therefore, synthetic compounds or biomolecules that modulate Jak3 functions without having impairing effects on normal immune functions will be great value as drug to treat a wide variety of disorders mediated by Jak3. A few examples of diseases that can be treated through targeting Jak3 are listed in Table 1.

TABLE 1
Immune-mediated diseases/disorders where Jak3 is targeted.

| Condition | Rationale | Refs |
|---|---|---|
| Asthma, allergies | Jak3 is a key regulator of IgE-mediated mast cell | (8, 12) |
| | responses | |
| Type I diabetes | T-cell-mediated destruction of insulin-secreting | (13-15) |
| Hematopoietic malignancies | pancreatic B cells. Severe combined | |
| | immunodeficiency disease | |
| Transplantation | Acute lymphoblastic leukemia T-cell reactivity | (16-18) |
| | and cytokine induction in acute allograft rejection | (19, 20) |
| | Induction of T-cell hypo-reactivity | (21) |
| Thromboembolitic | Presence of active Jak3 in platelets | (22) |
| complications in | | |
| transplantation | | |
| Lou Gehrig disease | WHIP131, a Jak3 inhibitor prolongs patient | (22) |
| | survival | |

### NEW AVENUES TO DEVELOP JAK3 TARGETED DRUGS

### Exploitation of the Multi-Domain Architecture of Jak3 to Modulate its Functions;

There are several Jak3 inhibitors available that target kinase domain. A recently discovered orally active inhibitor of Jak3, CP-690,550 has shown promising results as therapeutic immunomodulator (23). In general, exploiting the nucleotide-binding site of kinases has long been an attractive strategy for drug design (24-26). Recent trends in pharmaceutical sector have shown successful outcomes by targeting kinases (27). For Jak3 as well, though it has multiple domains but most efforts have been concentrated on the kinase domain only.

Like other Jaks, Jak3 has a modular structure consisting of seven distinct domains. Though communication between domains is an important part of Jak functions but it is not fully understood. Using present invention it was shown that the communication between FERM and SH2 domain is essential for the physiological functions of Jak3 (28). Therefore the presence of different domains in Jak3 might offer alternative options to regulate its activity by using non-catalytic domains of the molecule. Apart for the kinase domain, the functions of other domains of Jak3 which include SH2-like domain (between JH3 and JH4) and the N-terminal FERM region can be utilized to modulate its functions.

Src homology 2 (SH2) domain are conserved region of approximately 100 amino acid residues which is required for multi-protein complex formation. Though devoid of kinase activity, these domains regulate the function of kinase domain through mediating the interaction of tyrosine kinases with cellular substrates. The interaction between proteins with SH2 domains and their binding partner is direct, specific and phosphotyrosine dependent. These interactions facilitate the recruitment of tyrosine kinase-associated proteins and activation of downstream signaling cascade (29). Peptide-binding studies of the SH2 domain of Src and Lck show a marked preference for the sequence Y(P)EEIE. Interestingly, rosmarinic acid (RosA) inhibits Y(P)EEIE binding to the SH2 domain of Lck. The inhibitory effect is further increased (3 fold) by the incorporation of a negatively charged amino acid, which indicates that non-phosphopeptides might be potent inhibitors of SH2 domains. Conversely, non-peptide inhibitors of the SH2 domain of Src have been designed, based on its crystal structure (29), and inhibitors of the SH2 domain of the adaptor protein Grb2 are available (29). Based on the existence of a hydrophobic region adjacent to the primary ligand-binding site of the SH2 domain of Grb2, hydrophobic groups have been designed that improve the affinity of the minimal sequence Y(P)IN, which is recognized by the SH2 domain of Grb2. The final product has an IC50 of 1.6 nM and is highly selective for the SH2 domain of Grb2 compared with many other SH2 domains. Homology modeling of the SH2 domain of Tyk2 has been reported based on sequence considerations (29). Interestingly, the putative SH2 domain of Tyk2 has a histidine residue instead of the conserved arginine and hence the binding specificity of the SH2 domain of Tyk2 for Y(P)-containing peptides might be different from other SH2 domains. Nevertheless, studies to elucidate the precise role of the SH2-like domain of Jaks are required to assist the development of novel inhibitory compounds. Using the technology claimed in the present application we showed that tyrosine phosphorylation of SH2 domain was not only essential for the activation of Jak3 but also for Jak3 interactions with other biomolecules (28), thus defining one of the roles of Jak3 SH2 domain.

The N-terminal region of Jak3 (w550 amino acids) is important for the binding of Jak3 not only to the cytoplasmic tail of the cytokine receptors but also to cytoskeletal proteins (28) and adapter protein (30). A minimal segment of w200 residues with cytokine-receptor-binding specificity has been identified in this region of Jak3. The N-terminal region of Jaks is suggested to possess a four-point-one ezrin/radixin/moesin (FERM) domain (31). In the three ERM proteins, the FERM domain is implicated in cell-cell communication and cell adhesion by mediating binding to the cytosolic parts of membrane proteins. The crystal structure of the moesin FERM domain masked by the C-terminal actin-binding tail of moesin (non-covalent complex) and that of the radixin FERM domain have been elucidated recently (32) (33), to reveal three subdomains that are arranged in a trigonal fashion to form several grooves and clefts. Each subdomain shows a different fold: ubiquitin fold (subdomain 1); acyl-CoA-binding-protein fold (subdomain 2); and phosphotyrosine-binding (PTB)-domain fold (subdomain 3). Selective Y(P)-mimetics have been designed for the PTB domain of Shc. However, in the absence of structural information, it is challenging to attempt to design drugs aimed at the N-terminal region of JAK3. Nevertheless, the N-terminal region of Jak3 is an attractive drug target because it appears to be involved in both receptor binding and the maintenance of kinase integrity (34). Thus, a potent inhibitor of Jak3 could, ideally, lead to kinase inhibition and dissociation from, the receptor. Notably, staurosporine, a prototype kinase inhibitor, decreases the binding of Jak3 to the gc (34). The present invention offers a system to screen FERM domain based inhibitors of Jak3 functions.

### DETAILED DESCRIPTION OF THE PRESENT INVENTION

Disclosed in the present invention are screening tools to identify not only Jak3 inhibiting compounds and biomolecules but also those compounds and biomolecules which may not interfere with Jak3 activations but still interact with Jak3 and have implications in treatment of diseases where normal functions of Jak3 are required but the disease causing functions of Jak3 are undesirable. Given below is an example (and detailed in the enclosed publication).

Though villin or gelsolin proteins do not interact with the kinase-domain of Jak3 but using the present inventions it is shown that villin interacts with Jak3 and these interactions are important for the migration of cells expressing Jak3. Cell-migration of cancer cells is important for their metastasis thus using present technology it is possible to disrupt this interactions and impair cancer cells metastasis.

As described in the attached two publications (Mishra et. al. 2012, and Mishra and Kumar 2014), the present invention comprises purified functionally active full-length Jak3 proteins (e.g. FIG. 1A; Mishra et. al. 2012) which do not have any other human or other eukaryotic proteins impurities. Additionally this also comprises one or a plurality of seven purified functionally active domains of Jak3 proteins. The attached article describes the auto-phosphorylation of Jak3 (e.g. FIG. 1B; Mishra et. al. 2012, with no external substrate). The articles also describe trans-phosphorylation of other proteins/biomolecules by functionally active Jak3 (FIG. 1D, FIG. S1; Mishra et. al. 2012, and FIG. 1B, FIG. 1F; Mishra and Kumar 2014). We also describe Jak3 interacting compounds (e.g. FIG. 1C; Mishra et. al. 2012) or biomolecules (e.g. FIG. 1F; Mishra et. al. 2012, and FIG. S3; Mishra and Kumar 2014) that interact with Jak3 either through Jak3's kinase-domain or other specific domains of Jak3 (FIG. 1J-K, FIG. S2A-B; Mishra et. al. 2012, and FIG. 1G-I; Mishra and Kumar 2014) in a system which does not have any other human or other eukaryotic proteins impurities. The screening kit also comprises a method for determining the mechanism of action and effectiveness for Jak3 interacting drugs (e.g. FIG. 1C; Mishra et. al. 2012).

We have also developed and optimized the process of producing and purifying (a) functionally active full-length Jak3, (b) each individual domains of Jak3, (c) different combinations of the seven domains of Jak3 proteins. An example of the essential parts of the process to produce full length Jak3 is given below;

One of the main problems in producing full length biologically active Jak3 is the optimized distance between GST and Jak3 open reading frames in the DNA construct. Using a series of internal deletion mutants we have optimized the distance between GST and Jak3 that results in maximum production of Jak3. Since Jak3 is a high molecular weight protein, its production is not supported by commercially available growth media. We have optimized the ingredient compositions and concentrations for optimal production of Jak3 proteins. Since Jak3 is a mammalian protein tyrosine kinase, it is essential to produce the Jak3 protein which is free from other mammalian and other eukaryotic protein which may interferes with its activity giving rise to a high background thereby necessitating the use of radioactive methods of detection of activity. We have developed a method using cold lyses of cells bearing recombinant Jak3 proteins with strict prohibition of sonication and/or French-press. This results in stable and biologically active full length Jak3 which do not have any other human or other eukaryotic proteins impurities. To increase the yield of purification, we have optimized the binding between GST (Glutathione S-transferase) affinity beads and GST-Jak3 proteins under cold conditions in a specialized buffer containing cocktails of protease- and phosphatase inhibitors. To maintain the post-purification stability and functionality of Jak3, we describe specific protocol for post-purification processing of the protein for biological stability and functions that involves steps for dialysis, lyophilization, storage, and reconstitution.

On the screening part, once the most potent lead compound or the biomolecules are identified, we describe a process for determining the recommended effective doses (for the effectiveness). The essential parts of the processes determine Jak3 proteins level in patient (wherever biopsy samples are available) followed by determining effective IC50 and LD 50 using culture of the patient's biopsy tissue or using human established cell lines. Determination of effective doses is based on the plasma level of the drug using the lead compound in the range between 1050 and LD 50 correlated with amelioration of the disease symptoms.

### SUMMARY

Currently available Jak3 inhibitor screening technologies use only the tyrosine kinase domain of Jak3. However, the screening technology presented in this application not only utilizes the full-length of Jak3 but also all the domains individually or in combination. An example of the innovative advantages of present invention compared to commercially available systems is summarized in Table 2 below:

TABLE 2
An example of side by side comparison of currently available assay systems and
assay system disclosed in the present invention for Jak3 inhibitors only

| Commercially available assay using | |
|---|---|
| JAK3 kinase domain | Novel assay under present invention |
| 1. Commercially available Jak3 | Our full-length Jak3 has very low level of auto- |
| Kinase domain has high level of auto- | (tyrosine) phosphorylation. (illustrated in the |
| (tyrosine) phosphorylation | attached publication FIG. 1A top panel lane 1) |
| 2. For the reason mentioned in (1) | Our full-length JAK3 gives very low level of |
| commercially available Jak3 gives | background. |
| high level of background. | |
| 3. To check the activity of the enzyme | To check the activity of full-length Jak3 user does |
| user has to use an external substrate (a | need to use an external substrate (illustrated in the |
| commercially available peptide) | attached publication FIG. 1B) (28). |
| 4. For the reason mentioned in (1) | For the reason mentioned in (1) auto-phosphorylation |
| auto-phosphorylation mediated by | mediated by JAK3 can be assayed using our full- |
| JAK3 cannot be assayed using | length JAK3 (illustrated in the attached publication |
| commercially available JAK3 kinase | FIG. 1B). |
| domain. | |
| 5. Kinase activity can be measured | Kinase activity (both auto- and trans-phosphorylation |
| only by radioactive method in order to | can be measured by user friendly non-radioactive |
| have low level of background. | methods (illustrated in the attached publication FIGS. |
| | 1B and 1D). |
| 6. For the reason mentioned in (1) and | For the reason mentioned in (1) and (2), the JAK3 |
| (2), the JAK3 inhibitor CP-690550 | inhibitor CP-690550 showed a dose dependent |
| showed very minimal inhibition only | inhibition of JAK3 auto-phosphorylation at a |
| at a concentrations of 50 μM (data | concentration range between 5-500 nM (illustrated in |
| shown below in FIG. 1 of this | the attached publication FIG. 1C) with IC₅₀ = 128 nM. |
| application) | |

In summary, the present Jak3 (or its domain) assay system comprises the use of full-length Jak3 (or its individual domain or combination of domains), different from currently available in that only the active site of Jak3 is used. The advantages of our novel screening system include;

(a) significantly lower background noise, (b) higher sensitivity, (c) no need for the external substrate, (d) no need to use the radioactive method, and (e) capable of measuring auto-(tyrosine) phosphorylation.

The immediate and long term goals of the present assay system is to augment the discovery of potential new drug candidates through screening Jak3 interacting compounds, biomolecules, and inhibitors that cannot be detected by other available assays systems. Overall the application of present inventions is intended for medicinal chemists and other drug company to develop improved and more effective modulator of Jak3 to be used in the treatment of wide variety of Jak3-mediated diseases.

### BRIEF DESCRIPTION OF THE FIGURE

FIG. 1. Dose effect of CP-690550 on auto-phsophorylation of commercially available Jak3 kinase domain. Changes in tyrosine auto-phosphorylation of Jak3-kinase domain were detected in the presence or absence of different concentrations of Jak3-inhibitor CP-690505 using a 96-well multiplate coated with Jak3-kinase domain using pY-antibody through ELISA. Absorbance was measured at 450 nm in an ELISA plate reader.

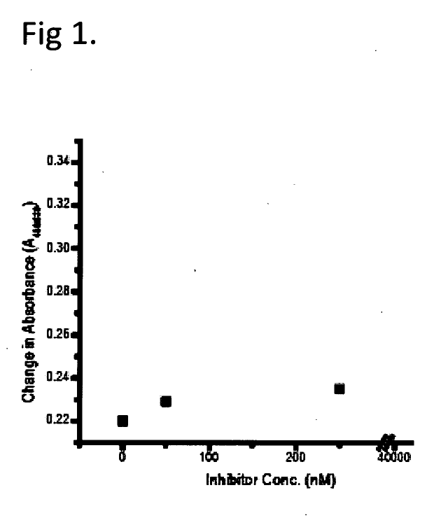

1. A screening kit to identify drugs for Asthma, allergies, Type I diabetes, Hematopoietic malignancies, Transplantation, Thromboembolic complications in transplantation, Lou Gehrig disease, Crohn's disease, Ulcerative colitis, Leukemia, T-cell and B-cell lymphomas, and colorectal cancer using inhibitors for Jak3.

2. The screening kit according to claim 1 which comprises purified functionally active full-length Jak3 proteins which do not have any other human or other eukaryotic proteins impurities.

3. The screening kit according to claim 1 which comprises one or a plurality of seven purified functionally active domains of Jak3 proteins which do not have any other human or other eukaryotic proteins impurities.

4. A method of using the screening kit according to claim 1 for assaying the auto-phosphorylation of Jak3 (with no external substrate) in a system which does not have any other human or other eukaryotic proteins impurities.

5. A method of using the screening kit according to claim 1 for identifying Jak3 interacting compounds or biomolecules that may interact with Jak3 either through Jak3's kinase-domain or any other specific domains of Jak3 in a system which does not have any other human or other eukaryotic proteins impurities.

6. A method of using the screening kit according to claim 1 for determining the mechanism of action and effectiveness for Jak3 interacting drugs.

7. A method of using the screening kit according to claim 1 for comparing the binding affinity of several drugs simultaneously that interacts with Jak3.

8. The screening kit according to claim 1 which comprises composition and cocktails of solutions required in the screening kit.

9. A process for determining effective doses of compounds, proteins/biomolecules identified using the screening kit according to claim 1.

10. A process of producing and purifying, (a) functionally active full-length Jak3, (b) each individual domains of Jak3, (c) different combinations of the seven domains of Jak3 proteins which does not have any other human proteins impurities wherein the process comprises the following elements: Optimized nucleotide distance between the codons of GST's and Jak3 (or its domains) for optimal expression of recombinant proteins; use of formulated enriched media for increased expression of recombinant proteins; cold lyses of cells with strict prohibition of sonication and or French-press; optimized binding conditions between GST affinity beads and GST-Jak3 under cold conditions in a specialized buffer containing a cocktails of protease and SHP2- and PTP1B-specific phosphatase inhibitors; post-purification processing of the protein for biological stability and functions that involves steps for dialysis, lyophilization, storage, and reconstitution.
